(19) United States
(12) Patent Application Publication
(10) Pub. No.: US 2025/0366768 A1
(43) Pub. Date: Dec. 4, 2025

(54) NONINVASIVE SYSTEMS AND METHODS FOR QUANTIFYING NEURAL SYNCHRONY IN THE COCHLEAR NERVE AND CORRELATING THE QUANTIFIED NEURAL SYNCHRONY WITH TEMPORAL RESOLUTION ACUITY AND SPEECH PERCEPTION OUTCOMES MEASURED IN QUIET AND IN NOISE

(71) Applicants: Ohio State Innovation Foundation, Columbus, OH (US); McMaster University, Hamilton (CA)

(72) Inventors: Shuman He, Columbus, OH (US); Ian C. Bruce, Hamilton (CA)

(21) Appl. No.: 19/227,614

(22) Filed: Jun. 4, 2025

Related U.S. Application Data
(60) Provisional application No. 63/655,820, filed on Jun. 4, 2024.

(51) Int. Cl. A61B 5/38; A61B 5/388; A61N 1/36

(52) U.S. Cl. CPC A61B 5/38; A61B 5/388; A61N 1/36038; A61N 1/36171

(57) ABSTRACT

Disclosed herein are of systems, methods, and computer-program products of determining a peripheral neural synchrony value based on eCAPS and correlating the peripheral neural synchrony value with temporal resolution acuity and speech perception outcomes measured in quiet and in noise in the patient.

### CROSS REFERENCE TO RELATED APPLICATION

This application claims priority to and benefit of U.S. provisional patent application Ser. No. 63/655,820 filed Jun. 4, 2024, which is fully incorporated by reference and made a part hereof.

### GOVERNMENT SUPPORT CLAUSE

This invention was made with government support under grant/contract numbers 1R01 DC016038, 1R01 DC017846, R21 DC019458 awarded by the National Institutes of Health. The government has certain rights in the invention.

### BACKGROUND

As shown in FIG. 1, cochlear implants convert sounds (picked up by a microphone) into electrical representations (in the sound processor) which are transmitted through the head and stimulate nerves surrounding the cochlea. Cochlear implants bypass the normal hearing pathway (i.e. middle car bones and inner ear hair cells), which is usually not functional when somebody is deaf. However, cochlear implants still rely on the cochlear nerve to be functional in order to carry the signal to the brain for further processing and interpretation (as shown in FIG. 2).

In humans, there is no way to directly evaluate how well the cochlear nerve (CN) functions. However, as shown in FIG. 3, user defined stimuli can be sent through one electrode of a cochlear implant to stimulate the surrounding neurons. The neurons then respond, and the electrical response can be recorded by a neighboring electrode. The neural response is called the electrically-evoked compound action potential (eCAP). The eCAP response can be characterized as an indirect measure of neural function. Additional information about eCAPS and measuring neural response using eCAPS can be found in US PG-PUB US-2023-0200712-A1, SYSTEMS AND METHODS FOR DETECTING THE PRESENCE OF ELECTRICALLY EVOKED COMPOUND ACTION POTENTIALS (eCAPS), ESTIMATING SURVIVAL OF AUDITORY NERVE FIBERS, AND DETERMINING EFFECTS OF ADVANCED AGE ON THE ELECTRODE-NEURON INTERFACE IN COCHLEAR IMPLANT USERS, published Jun. 29, 2023, which is fully incorporated by reference and made a part hereof.

While cochlear implant users can achieve excellent listening outcomes in quiet, speech recognition in background noise remains a significant challenge. Studies in acoustic hearing have demonstrated that human listeners with degraded neural synchrony at the level of the CN (such as patients with auditory neuropathy spectrum disorder), experience excessive difficulties in understanding speech in background noise. Additionally, it has been shown that neural synchrony at the level of the CN is a strong predictor of speech recognition in noise for both young and elderly listeners with acoustic hearing.

In acoustic hearing, discharge synchronization of CN fibers has been shown to play a critical role in neural representation of speech sounds presented in noise in animal models (e.g., Delgutte & Kiang, 1984; Heeringa & Koppl, 2022; Sachs et al., 1983). Simulation results from computational models demonstrated the importance of synchronized neural firing from CN fibers for robust encoding of consonants in spectro-temporally modulated background noises (Bruce et al., 2013; Viswanathan et al., 2022). These simulation results also showed that poor neural synchrony in the CN (i.e., peripheral neural synchrony) results in smeared neural representation of temporal envelope cues, which leads to deficits in processing these cues (Zeng et al., 2005; Zeng et al., 1999). Aligned with these results from animal models and computational simulations, listeners with poor peripheral neural synchrony (e.g., patients with auditory neuropathy spectrum disorder and elderly listeners) have temporal processing deficits and show excessive difficulties in understanding speech in noise (e.g., Harris et al., 2021; Kraus et al., 2000; Rance, 2005; Zeng et al., 2005). Overall, these results demonstrate the importance of peripheral neural synchrony for temporal processing and speech perception in noise in acoustic hearing.

Therefore, systems and methods are desired to overcome challenges in the art, some of which are described above. More specifically, there is a need for systems and methods for assessing neural synchrony at the level of the CN in cochlear implant (CI) users with recordings of the electrically evoked compound action potential.

### SUMMARY

Disclosed and described herein are noninvasive systems and methods for quantifying neural synchrony in the cochlear nerve (i.e., peripheral neural synchrony) in cochlear implant (CI) users, which allows for evaluating this physiological phenomenon in human CI users. By quantifying peripheral neural synchrony, it can be correlated with temporal resolution acuity and speech perception outcomes measured in quiet and in noise in post-lingually deafened adult CI users.

The disclosed systems and methods allow for assessing neural synchrony of a population of electrically stimulated CN fibers by quantifying the trial-to-trial phase coherence in the summated activity to electrical stimulation using electrophysiological measures of the eCAP.

In one aspect, methods of determining a peripheral neural synchrony value based on eCAPS and correlating the peripheral neural synchrony value with temporal resolution acuity and speech perception outcomes are disclosed. One embodiment of the method comprises (a) providing a stimulus at a first of electrode of a cochlear implant (CI) of a patient, wherein the stimulus is set at or below a comfort level of the patient; (b) measuring eCAP at a second electrode of the CI of the patient, said eCAP based on a neural response to the stimulus; (c) repeating steps (a) and (b) sequentially a plurality of times for a plurality of electrodes of the CI to obtain a plurality of eCAP measurements; (d) evaluating a phase coherence among the plurality of eCAP measurements to determine a peripheral neural synchrony value; and (e) correlating the peripheral neural synchrony value with temporal resolution acuity and speech perception outcomes measured in quiet and in noise in the patient.

Additional aspects disclosed herein include systems for implementing the disclosed methods; and non-transitory computer program products with computer-executable code sections stored thereon for implementing the disclosed methods.

Additional advantages will be set forth in part in the description which follows or may

be learned by practice. The advantages will be realized and attained by means of the elements and combinations particularly pointed out in the appended claims. It is to be understood that both the foregoing general description and the following detailed description are exemplary and explanatory only and are not restrictive, as claimed.

### BRIEF DESCRIPTION OF THE DRAWINGS

The accompanying drawings, which are incorporated in and constitute a part of this specification, illustrate embodiments and together with the description, serve to explain the principles of the methods and systems.

FIG. 1 is an image illustrating an example of a cochlear implant.

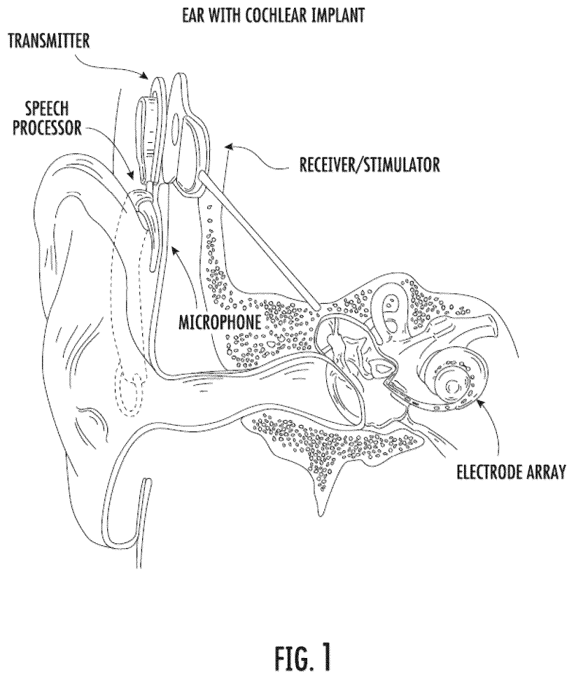

FIG. 2 is an image illustrating how cochlear implants still rely on the cochlear nerve to be functional in order to carry the signal to the brain for further processing and interpretation.

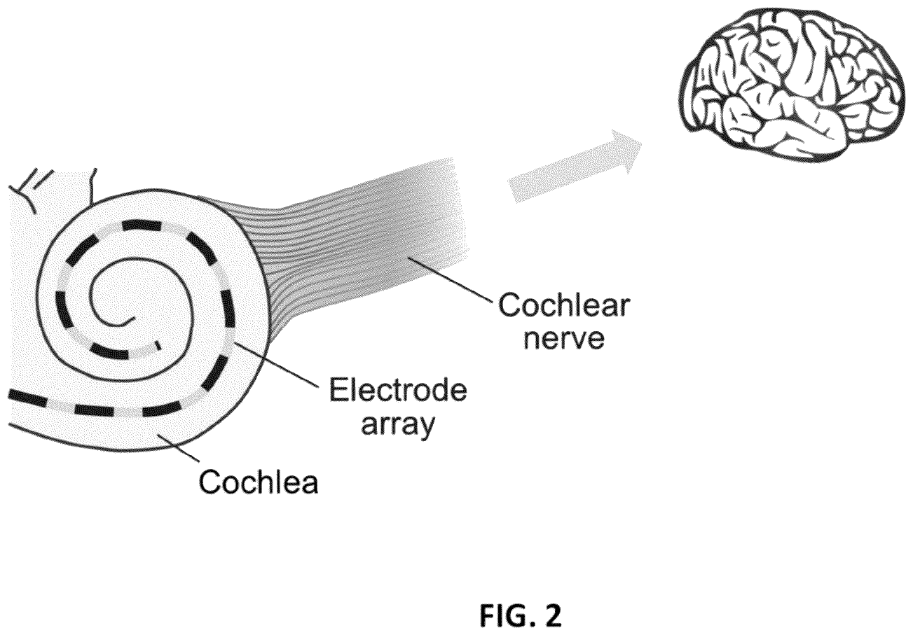

FIG. 3 is an illustration of how user defined stimuli can be sent through one electrode of a cochlear implant to stimulate the surrounding neurons, with the compound neural response (i.e., eCAP) being recorded by a different electrode of the cochlear implant.

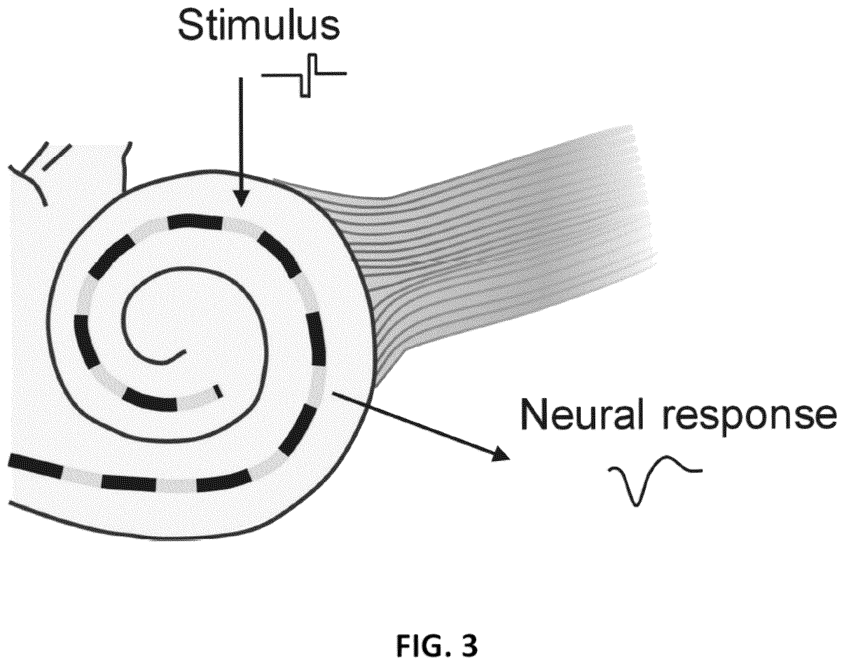

FIG. 4 illustrates a method of determining a peripheral neural synchrony value based on eCAPS and correlating the peripheral neural synchrony value with temporal resolution acuity and speech perception outcomes measured in quiet and in noise in the patient.

FIG. 5 illustrates means and standard deviations of phase-locking values measured at four electrode locations in 24 adult cochlear implant users (27 ears).

FIG. 6 illustrates phase-locking values (PLV) and psychophysical gap detection thresholds (GDT) measured at two electrode locations in each of 23 implanted ears of 20 participants. Lines connect the data measured at the two electrode locations tested in the same ear.

FIG. 7 illustrates stimulation levels and psychophysical gap detection thresholds measured at two electrode locations in each of 23 implanted ears of 20 participants. Lines connect the data measured at the two electrode locations tested in the same ear.

FIG. 8 illustrates Consonant-Nucleus-Consonant (CNC) word scores measured in quiet and in two noise conditions as a function of the phase-locking value averaged across electrode locations for 23 adult cochlear implant users (26 ears). The best fit line across all 26 data points is illustrated with a solid line. The results from Pearson's correlation analysis are also provided in each panel.

FIG. 9 illustrates change in Consonant-Nucleus-Consonant (CNC) word scores with the addition of background noise as a function of the phase-locking value averaged across electrode locations. The best fit line across all 26 data points is illustrated with a solid line. The results from Pearson's correlation analysis are also provided in each panel.

FIG. 10 illustrates an exemplary computing device that can be used according to embodiments described herein.

### DETAILED DESCRIPTION

Before the present methods and systems are disclosed and described, it is to be understood that the methods and systems are not limited to specific synthetic methods, specific components, or to particular compositions. It is also to be understood that the terminology used herein is for the purpose of describing particular embodiments only and is not intended to be limiting.

As used in the specification and the appended claims, the singular forms “a,” “an” and “the” include plural referents unless the context clearly dictates otherwise. Ranges may be expressed herein as from “about” one particular value, and/or to “about” another particular value. When such a range is expressed, another embodiment includes from the one particular value and/or to the other particular value. Similarly, when values are expressed as approximations, by use of the antecedent “about,” it will be understood that the particular value forms another embodiment. It will be further understood that the endpoints of each of the ranges are significant both in relation to the other endpoint, and independently of the other endpoint.

“Optional” or “optionally” means that the subsequently described event or circumstance may or may not occur, and that the description includes instances where said event or circumstance occurs and instances where it does not.

Throughout the description and claims of this specification, the word “comprise” and variations of the word, such as “comprising” and “comprises,” means “including but not limited to,” and is not intended to exclude, for example, other additives, components, integers or steps. “Exemplary” means “an example of” and is not intended to convey an indication of a preferred or ideal embodiment. “Such as” is not used in a restrictive sense, but for explanatory purposes.

Disclosed are components that can be used to perform the disclosed methods and systems. These and other components are disclosed herein, and it is understood that when combinations, subsets, interactions, groups, etc. of these components are disclosed that while specific reference of each various individual and collective combinations and permutation of these may not be explicitly disclosed, each is specifically contemplated and described herein, for all methods and systems. This applies to all aspects of this application including, but not limited to, steps in disclosed methods. Thus, if there are a variety of additional steps that can be performed it is understood that each of these additional steps can be performed with any specific embodiment or combination of embodiments of the disclosed methods.

As will be appreciated by one skilled in the art, the methods and systems may take the form of an entirely hardware embodiment, an entirely software embodiment, or an embodiment combining software and hardware aspects. Furthermore, the methods and systems may take the form of a computer program product on a computer-readable storage medium having computer-readable program instructions (e.g., computer software) embodied in the storage medium. More particularly, the present methods and systems may take the form of web-implemented computer software. Any suitable computer-readable storage medium may be utilized including hard disks, CD-ROMs, optical storage devices, or magnetic storage devices.

Embodiments of the methods and systems are described below with reference to block diagrams and flowchart illustrations of methods, systems, apparatuses and computer program products. It will be understood that each block of the block diagrams and flowchart illustrations, and combinations of blocks in the block diagrams and flowchart illustrations, respectively, can be implemented by computer program instructions. These computer program instructions may be loaded onto a general-purpose computer, special purpose computer, or other programmable data processing apparatus to produce a machine, such that the instructions which execute on the computer or other programmable data processing apparatus create a means for implementing the functions specified in the flowchart block or blocks.

These computer program instructions may also be stored in a computer-readable memory that can direct a computer or other programmable data processing apparatus to function in a particular manner, such that the instructions stored in the computer-readable memory produce an article of manufacture including computer-readable instructions for implementing the function specified in the flowchart block or blocks. The computer program instructions may also be loaded onto a computer or other programmable data processing apparatus to cause a series of operational steps to be performed on the computer or other programmable apparatus to produce a computer-implemented process such that the instructions that execute on the computer or other programmable apparatus provide steps for implementing the functions specified in the flowchart block or blocks.

Accordingly, blocks of the block diagrams and flowchart illustrations support combinations of means for performing the specified functions, combinations of steps for performing the specified functions and program instruction means for performing the specified functions. It will also be understood that each block of the block diagrams and flowchart illustrations, and combinations of blocks in the block diagrams and flowchart illustrations, can be implemented by special purpose hardware-based computer systems that perform the specified functions or steps, or combinations of special purpose hardware and computer instructions.

The present methods and systems may be understood more readily by reference to the following detailed description of preferred embodiments and the Examples included therein and to the Figures and their previous and following description.

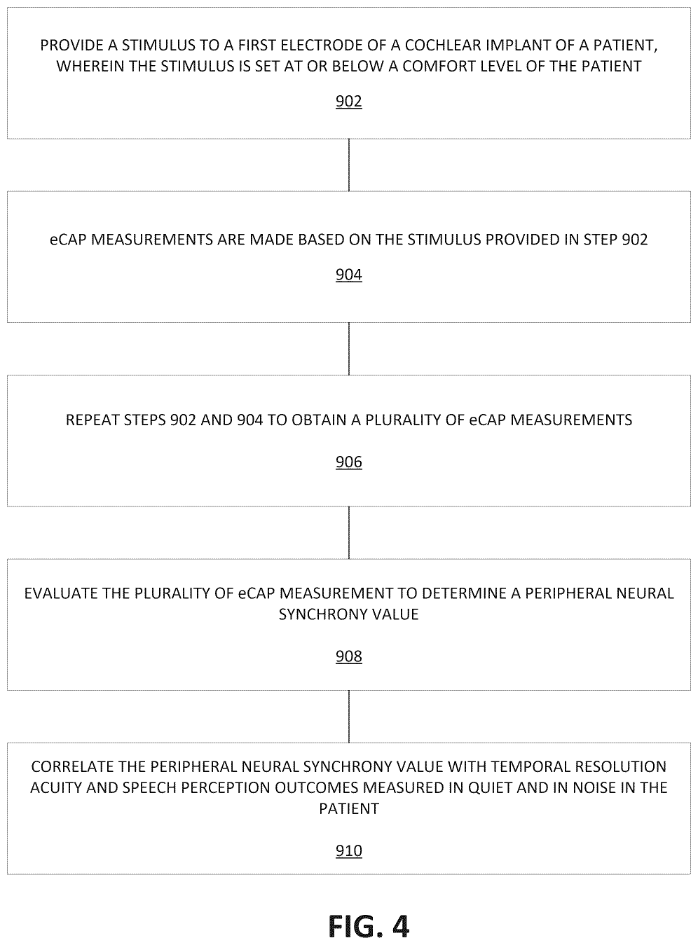

FIG. 4 is a flowchart for an exemplary method of determining a peripheral neural synchrony value based on eCAPS; and correlating the peripheral neural synchrony value with temporal resolution acuity and speech perception outcomes measured in quiet and in noise in the patient. At step 902, a stimulus is provided to a first electrode of a cochlear implant of a patient, wherein the stimulus is set at or below a comfort level of the patient. For eCAP recording, the stimulus may comprise, as an example, a charge-balanced, cathodic-leading, biphasic pulse with an interphase gap of 7 μs and a pulse phase duration of 25 μs/phase. For measuring psychophysical gap detection 165 threshold (GDT), the stimulus may be a train of biphasic pulses with the same characteristics as those of the single-pulse stimulus that was presented for 500 ms at a stimulation rate of 900 pulses per second (pps) per channel. It is to be appreciated that these parameters are exemplary only and can be adjusted for different studies and/or patients. For both measures, the stimulus was delivered to individual CI electrodes in a monopolar-coupled stimulation mode via an N6 sound processor (Cochlear Ltd, Macquarie, NSW, Australia) interfaced with a programming pod, though other devices and/or methods can be used for delivering the stimulus.

The maximum comfortable level (i.e., the C level) for each type of stimulus was determined using an ascending procedure. In this procedure, study participants were instructed to use a visual loudness rating scale [scale of 1-10, where 1 is “barely audible” and 10 is “very uncomfortable”] to indicate when the sound reached the maximum comfort level (rating of 8). Stimulation was first presented at a relatively low level and gradually increased in steps of 5 clinical units (CUs) until a loudness rating of 7 was reached. Then, stimulation was increased in steps of 1-2 CUs until a rating of 8 (“maximal comfort”) was reached. It is to be appreciated that the CU is used for Cochlear Nucleus (Cochlear Ltd, Macquarie, NSW, Australia) devices only, and that other units and/or scales can be used for adjusting the C level for other devices and/or methods. The C level was measured for each type of stimulation delivered to each test CI electrode for each participant.

For the single-pulse stimulation used for eCAP recording, the stimulus was presented to individual CI electrodes using, for example, the “Stimulation Only” mode in the Advanced Neural Response Telemetry (NRT) function implemented in the Custom Sound EP (v. 6.0) commercial software (Cochlear Ltd, Macquarie, NSW, Australia) software. Due to the challenge of reliably rating loudness for an extremely brief single pulse, the C level was determined for a group of five pulses presented at 15 Hz. This is a standard clinical practice for determining the C levels during the programming process. As noted above, it is to be appreciated that different devices, methods and/or software can be used to provide the stimulus.

Returning to FIG. 4, at step 904, eCAP measurements are made based on the stimulus provided in 902. The eCAP recordings were obtained using, for example, the NRT function implemented in the Custom Sound EP (v. 6.0) commercial software (Cochlear Ltd, Macquarie, NSW, 222 Australia), though it is contemplated that different devices, methods and/or software can be used to obtain the eCAP recordings. The eCAP was measured at individual CI electrode locations using a two-pulse forward-masking-paradigm, though other artifact rejection techniques can be used to minimize artifact contaminations in eCAP recordings. In the two-pulse forward-masking-paradigm, the masker and the probe pulse were presented to the test electrode at the participants' C level and 10 CUs below the C level, respectively. The stimulation was presented 400 times using a slow probe rate (e.g., 15 Hz) to minimize the potential effect of long-term adaptation on the eCAP. The number of trials was chosen to be 400 because preliminary analyses indicated that this number of trials could ensure accurate estimation of neural synchrony in the CN while maintaining a feasible recording time. Results of previous studies have demonstrated that using electrophysiological results measured at single CI electrode locations to correlate with auditory perception outcomes in CI users can lead to inaccurate, if not wrong, conclusions. In comparison, the averaged results across multiple testing electrode locations provide a better representation of overall neural function than the result measured at any individual CI electrode locations. Based on these results, four electrodes across the electrode array were tested for each participant to get an estimate of overall CN function while maintaining a feasible testing time. The default testing electrodes in this example were electrodes 3, 9, 15, and 21. Though these electrodes are referred to herein, it is to be appreciated that in other instances different electrodes may be used and different devices having different electrodes may also be used. Alternate electrodes were tested in cases where there was an open-or short-circuit at the default electrode locations. The electrodes tested for each participant are listed in Table 1.

TABLE 1
Demographic information of all study participants,

| | | | | Internal device | | Electrodes | Electrodes | Speech |
|---|---|---|---|---|---|---|---|---|
| Participant | | Ear | Age | and electrode | Etiology of | tested | tested | scores |
| ID | Sex | tested | (years) | array | hearing loss | for PLV | for GDT | Included |
| A1 | M | L | 60s | CI512 | SHL | 4, 6, 9, 12 | 4, 9 | * |
| A2 | M | L | 60s | CI512 | Meniere's | 3, 9, 12, 15 | 3, 18 | * |
| A3 | F | L | 60s | CI24RE (CA) | Hereditary | 3, 9, 15, 21 | 3, 15 | * |
| A3 | F | R | 60s | CI24RE (CA) | Hereditary | 3, 9, 15, 21 | 3, 21 | * |
| A4 | F | L | 30s | CI24RE (CA) | Trauma | 3, 9, 15, 21 | 9, 21 | * |
| A5 | F | L | 50s | CI532 | Unknown | 4, 9, 15, 21 | 4, 21 | * |
| A5 | F | R | 50s | CI24RE (CA) | Unknown | 3, 9, 15, 21 | 9, 15 | * |
| A6 | M | R | 60s | CI522 | Trauma | 6, 9, 18, 21 | 9, 15 | * |
| A7 | M | R | 30s | CI24RE (CA) | Hereditary | 3, 9, 15, 21 | 3, 15 | * |
| A8 | F | R | 50s | CI24RE (CA) | Hereditary | 3, 12, 15, 21 | 3, 21 | * |
| A9 | F | R | 60s | CI532 | Unknown | 3, 9, 15, 20 | 3, 21 | * |
| A10 | M | R | 70s | CI532 | Trauma | 3, 9, 15, 21 | | * |
| A11 | F | L | 70s | CI422 | Noise | 4, 9, 15, 20 | 9, 20 | * |
| A12 | M | L | 60s | CI632 | Unknown | 3, 9, 15, 21 | 3, 21 | * |
| A12 | M | R | 60s | CI532 | Unknown | 3, 9, 15, 20 | 3, 20 | * |
| A13 | F | L | 70s | CI24RE (CA) | Autoimmune | 3, 7, 12, 18 | 3, 18 | * |
| A14 | M | L | 60s | CI532 | AN | 4, 9, 15, 21 | 9, 15 | * |
| A15 | F | R | 80s | CI532 | Hereditary | 3, 7, 10, 17 | 7, 17 | * |
| A16 | F | L | 30s | CI532 | Unknown | 3, 9, 15, 21 | | * |
| A17 | F | L | 50s | CI532 | Unknown | 3, 9, 15, 21 | 9, 21 | * |
| A18 | F | L | 70s | CI622 | Unknown | 6, 9, 15, 21 | | * |
| A19 | M | R | 80s | CI632 | Unknown | 3, 9, 15, 21 | 3, 9 | * |
| A20 | M | R | 50s | CI632 | SHL | 3, 9, 15, 21 | | * |
| A21 | F | L | 50s | CI632 | Unknown | 3, 15, 18, 21 | 15, 18\| | * |
| A22 | F | R | 70s | CI622 | Unknown | 3, 9, 15, 21 | 3, 15 | * |
| A23 | M | L | 50s | CI532 | Usher | 3, 9, 15, 21 | 3, 9 | * |
| A24 | M | L | 70s | CI632 | Unknown | 3, 9, 15, 21 | 3, 9 | * |

CI24RE (CA),
Freedom Contour Advance electrode array;
SHL, sudden hearing loss;
AN, acoustic neuroma

Other parameters used to record the eCAP included a recording electrode located two electrodes away apically from the stimulating electrode except for electrode 21 which was recorded at electrode 19, a 122-μs recording delay, an amplifier gain of 50 dB, and a sampling rate of 20,492 Hz. It is to be appreciated that other parameters can be used to measure the eCAP for other devices and/or methods. Step 906 comprises repeating steps 902 and 904 to obtain a plurality of eCAP measurements.

Returning to FIG. 4, at step 908 the plurality of eCAP measurements are evaluated to determine a peripheral neural synchrony value. Peripheral neural synchrony at individual CI electrode locations was evaluated based on 400 individual sweeps (trials) of the eCAP. Neural synchrony was quantified using an index named the phase locking value (PLV) which is a measure of trial-to-trial phase coherence among the 400 eCAP sweeps. The PLV is a unitless quantity that ranges from 0 to 1. It quantifies the degree of synchrony in neural responses generated by target CN fibers across multiple stimulation/trials. The PLV is influenced by temporal jitter in spike firing of individual CN fibers, discharge synchronization across the population of activated CN fibers, and discharge synchronization of a group of activated CN fibers across multiple stimulations. A PLV of 0 means that the distribution of phase across trials is uniform (i.e., the responses across trials are uncorrelated). The PLV is 1 if phases across trials are perfectly correlated. As a result, larger PLVs indicate better/stronger neural synchrony in the CN. Mathematically, the PLV is the length of the vector formed by averaging the complex phase angles of each trial at individual frequencies obtained via time-frequency decomposition. Specifically, the PLV is calculated at a specific frequency and time window (i.e., frame) as:

$$PLV ( f , t ) = \frac{1}{400} {\sum }_{k = 1}^{400} \vert \frac{{F}_{k} ( f , t )}{\vert {F}_{k} ( f , t ) \vert} \vert$$

where Fk(f,t) is the spectral estimate (i.e., complex number representing the amplitude and phase of a sinusoid obtained from the short-time Fourier transform) of trial k at frequency f for the time window t. For this non-limiting study, the time-frequency decomposition was performed at six linearly spaced frequencies (788.2, 1576.3, 2364.4, 3152.6, 3941.0 and 4729.2 Hz) with Hanning Fast Fourier Transform tapers, a pad-ratio of 2 and a frame size of 26 samples (1268.8 μs) using the newtimef function (v. 2022.1) included in the MATLAB plugin EEGlab (Delorme & Makeig, 2004). It is to be appreciated that these are only exemplary numbers for a Cochlear Nucleus˜ devices. Other devices may require different numbers, which are contemplated within the scope of this disclosure. For each CI electrode tested in each participant, a single PLV was obtained by averaging PLVs calculated at six frequencies for six partially overlapped frames with an onset-to-onset interval of 48.8 us between two adjacent frames within a time window of 1561.6 μs. The use of six partially overlapped frames within the time window of interest allows for higher temporal resolution of the PLV, with PLV values in the early frames capturing the degree of synchrony in the low-latency spikes and values in the later frames being dependent on synchrony in the longer-latency spikes.

The parameters used in PLV calculation were selected based on morphological characteristics of the eCAP measured in human CI users and the sampling rate offered by CI manufacturers for eCAP recording. Specifically, the eCAP recorded in human CI users comprises of one negative peak (N1) within a time window of 0.2-0.4 ms after stimulus onset followed by a positive peak (P2) occurring around 0.6-0.8 ms.

As a result, the longest inter-peak latency of the eCAP in human listeners is 600 μs. Using this duration as the half width of the sinusoid included in Fast Fourier Transform analysis and the sampling rate of 20,492 Hz offered by Cochlear˜ Nucleus® device for measuring the eCAP, the frame size was determined to be 1268.8us in time which included 26 samples (1268.8/48.8 =26). As a result, six frames with an onset-to-onset interval of 48.8 us between two adjacent frames cover the entire recording window [(1561.6-1268.8)/48.8 =6]. This frame size in time also determines the lowest frequency (1/1268.8 us =788.1 Hz) used in the PLV calculation. The difference in the peak-to-baseline amplitude between the NI and the P2 peak of the eCAP and the difference in their widths indicate a complex spectrum instead of a single fundamental frequency. A Fast Fourier Transform analysis was conducted to determine the frequency components of the averaged eCAPs over 400 sweeps measured. Results showed that the harmonic frequency with one quarter of the amplitude measured at the fundamental frequency was 4482.6 Hz. As a result, the highest frequency used in PLV calculation was determined to be 4729.2 based on a spectral resolution of 788.1 Hz determined by the frame size in time. This frequency range (i.e., 788.1-4729.2 Hz) is higher than that used in Harris et al. (2018, 2021). This difference is caused by morphological differences between the eCAP and the compound action potential evoked by acoustic clicks.

Within-channel GDTs were measured at two electrode locations with different PLVs in each of 23 ears tested in 20 participants (see Table 1 for the electrodes tested in each car). Pulse trains with and without temporal gaps were presented in a three-alternative, forced-choice paradigm that incorporated a three-down, one-up adaptative strategy to estimate 79.4% correct on the psychometric function (Levitt, 1971). Non-limiting individual trials consisted of three consecutive 500-ms listening intervals separated in time by 500-ms silent intervals. The stimulus presented in two of the three listening intervals was a 500-ms pulse train without any interruption. The stimulus presented in the remaining listening interval, chosen at random, included a temporal gap centered at 250 ms of stimulation. The participant was asked to determine which of the three listening intervals included two sounds. Feedback on correct/incorrect choices was not provided to participants. The gap duration began at 64 ms and was shortened/lengthened based on the correctness of the participants' choice. The initial step size of the change in gap duration was 32 ms. This step changed by a factor of two after three consecutive correct responses or one incorrect response. The minimum and maximum gap durations permitted were 1 ms and 256 ms, respectively. The GDT was calculated as the average across two trials in which the mean gap duration over the last four (of twelve) reversals was calculated. It is to be appreciated that the above is a non-limiting example of one study, and that other values and parameters can be used for gap detection threshold.

Returning again to FIG. 4, at step 910 the peripheral neural synchrony value is correlated with temporal resolution acuity and speech perception outcomes measured in quiet and in noise in the patient. Speech perception performance was evaluated separately for each implanted car using Consonant-Nucleus-Consonant (CNC) word lists (Peterson & Lehiste, 1962) presented in quiet and in two noise conditions. All auditory stimuli were presented in a sound-proof booth via a speaker placed one meter in front of the subject at zero degrees azimuth. The target stimulus was always presented at 60 dB sound pressure level (SPL). For the noise conditions, speech-shaped noise was presented concurrently with the target stimulus at 50 dB SPL and 55 dB SPL to create signal-to-noise ratios of +10 dB and +5 dB, respectively.

Descriptive statistics of PLVs measured at different electrode locations, GDTs and CNC word scores measured in different testing conditions and the degree of noise effect on CNC word scores which was quantified as the difference in CNC word scores measured in quiet and in noise were calculated, including the overall mean and standard deviation. Effects of electrode location and stimulation level on the PLV were assessed using a Linear Mixed effect Model (LMM) with electrode location and stimulation level as fixed effects. The effect of the PLV on GDT was evaluated using an LMM with the PLV, the stimulation level used to measure the GDT and the stimulation used to measure the PLV as fixed effects. All LMMs used a correlated regression model with an unstructured correlation matrix to account for repeated observations per participant. Estimations were obtained using restricted maximum likelihood with Satterthwaite degrees of freedom. The Tukey's Honest Significant Difference (Tukey's HSD) method was used to adjust for multiple comparisons. The difference in GDT between results measured at the two electrodes with different PLVs or stimulation levels were assessed using paired sample t-tests. One-tailed Pearson product-moment correlation tests with Bonferroni correction for multiple comparisons were used to assess the association of the PLV with CNC word scores measured in different conditions (α=0.017), as well as with the change in CNC word score with competing background noise (i.e., CNC word score measured in quiet-CNC word score measured in noise, α=0.025). Using electrophysiological results measured at single CI electrode locations to correlate with auditory perception outcomes in CI users can lead to inaccurate conclusions (He et al., 2023). Therefore, for these correlation analyses, PLVs measured at all electrode locations were averaged together for each participant/ear to obtain an estimation of the overall peripheral neural synchrony within the cochlea and to minimize electrode-location related bias in study results. One-tailed Pearson product-moment correlation tests with Bonferroni correction for multiple comparisons were also used to assess the association between CNC word scores measured in quiet and the degree of noise effect on CNC word scores. The strength of correlation was determined based on values of the Pearson product-moment correlation coefficient (r). Specifically, weak, moderate, and strong correlations were defined as r values between 0 and 0.3 (0 and −0.3), between 0.3 and 0.7 (—0.3 and −0.7), and between 0.7 and 1.0 (−0.7 and −1.0), respectively.

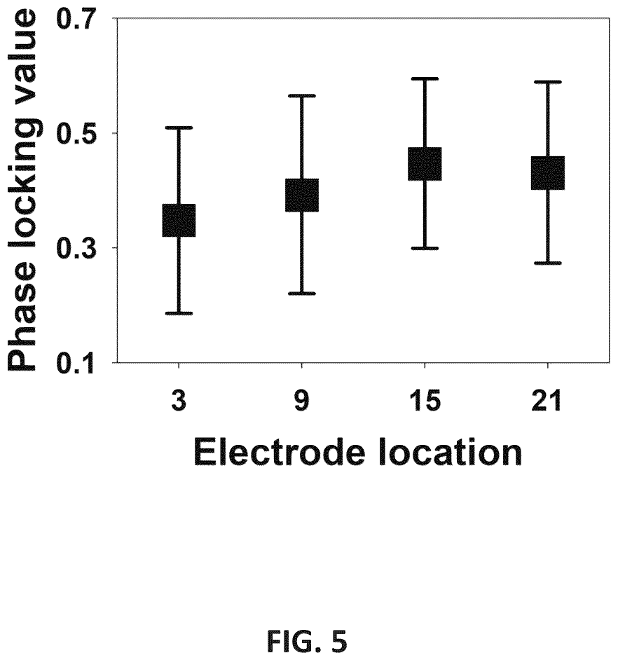

PLVs measured in this study ranged from 0.09 to 0.76 (mean: 0.55, SD: 0.22) across all electrodes tested. The means and standard deviations of PL Vs measured at each of the four electrode locations are shown in FIG. 5. The standard deviations at all four electrode locations demonstrate variations in the PLV in CI users. In addition, a trend for the mean PLV to be different across electrode locations is observed. PLVs measured at apical electrode locations (i.e., electrodes 15 and 21) appear to be larger than those measured at more basal electrode locations (i.e., electrodes 3 and 9). The results of the LMM showing a significant effect of electrode location on the PLV (xχ(3)2=17.11, p<0.001) after controlling for the significant effect of stimulation level on the PLV (t(101)=3.90, p<0.001). The results of pairwise comparisons showed that PLVs measured at electrode 15 were significantly larger than those measured at electrode 3(t(26.9)=−3.46, p=0.009) and electrode 9 (t(27.8)=−3.62, p=0.006). The differences in the PLV measured between other electrode pairs did not reach a statistical significance. Detailed results of pairwise comparisons are listed in Table 2.

TABLE 2
Results of pairwise comparisons for comparing phase locking
values measured at different electrode locations.

| Electrode | | Standard | Degree of | | |
|---|---|---|---|---|---|
| Pair | Estimate | Error | Freedom | T Ratio | p Value |
| E3 vs. E9 | −0.022 | 0.021 | 30.2 | −1.025 | .736 |
| E3 vs. E15 | −0.080 | 0.023 | 26.9 | −3.463 | .009 |
| E3 vs. E21 | −0.087 | 0.031 | 30.9 | −2.865 | .035 |
| E9 vs. E15 | −0.059 | 0.016 | 27.8 | −3.622 | .006 |
| E9 vs. E21 | −0.066 | 0.028 | 33 | −2.575 | .067 |
| E15 vs. E21 | 0.007 | 0.020 | 30.9 | −0.352 | .984 |

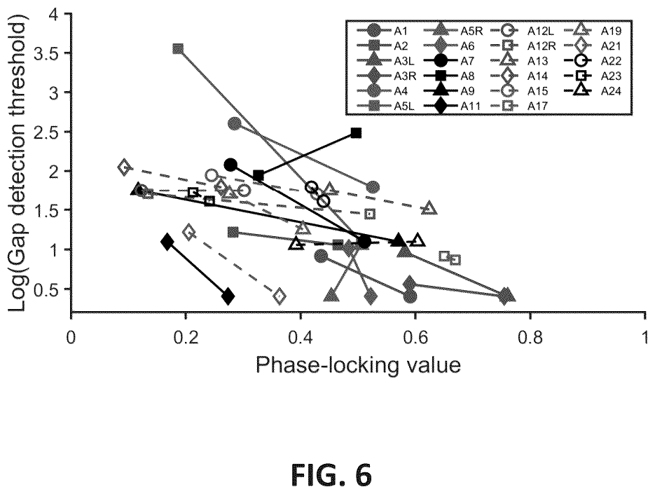

FIG. 6 shows psychophysical GDTs measured at two electrode locations per test ear in 20 participants (23 ears) as a function of the PLV. For the two CI electrode locations tested in each of 19 ears in 18 participants, smaller GDTs were always measured at the electrode locations with higher PLVs. Results measured in A8, A24 and the right ear of A5 showed an opposite pattern. In the left ear of A12, GDTs measured at the two CI electrode locations with different PLVs were the same (5.75 ms). The result of a paired-samples t-test showed that GDTs measured at the electrode locations with larger PLVs were statistically significantly smaller than those measured at electrode locations with smaller PLVs (t(22)=2.25, p=0.035). The results of the LMM showed a significant effect of the PLV on GDT (t(42)=−3.51, p=0.001) after controlling for the stimulation level of GDT (t(42)=−2.53, p=0.015) and the stimulation level of the PLV (t(42)=3.75, p<0.001), with larger PLVs leading to smaller GDTs.

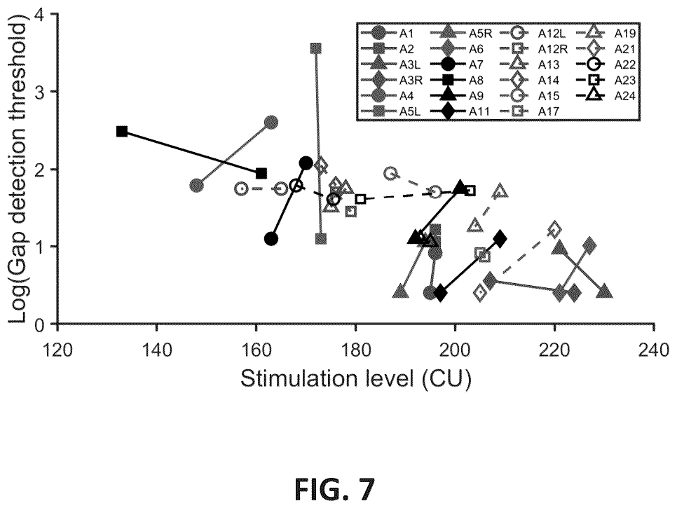

FIG. 7 shows psychophysical GDT results as a function of stimulation levels used to measure these GDTs. Smaller GDTs were measured at higher stimulation levels for the two CI electrode locations tested in each of 10 ears tested in 9 participants. Results measured in 11 ears tested in 11 participants showed an opposite relation between these two parameters. For A2, the stimulation levels used to measure GDTs at electrodes 3 and 18 were the same (196 CU). For A12, using different stimulation levels at electrodes 3 and 21 resulted in the same GDT (i.e., 5.75 ms). Overall, despite a significant stimulation level effect on GDT at a group level as shown in the LMM results, these data do not demonstrate a consistent association between stimulation level and GDT across participants, which differs from those shown in FIG. 6. Consistent with these observations, the result of a paired-samples t-test showed that GDTs measured at the electrode locations with higher stimulation levels were not significantly different from those measured at the electrode locations with lower stimulation levels (t(22)=−0.53, p=0.599).

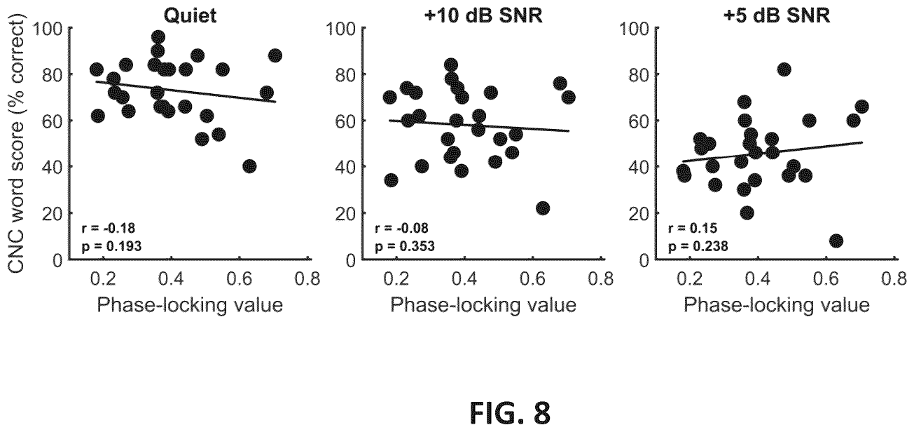

FIG. 8 shows CNC word scores measured in quiet and in two noise conditions as a function of the PLV for 23 participants (26 cars). There was substantial variability in CNC word scores measured in quiet (range: 40.0-96.0%, mean: 73.1%, SD: 13.3%) and in noise (+10 dB SNR: range: 22.0-84.0%, mean: 58.1%, SD: 15.8%; +5 dB SNR: range: 8.0-82.0%, mean: 45.6%, SD: 15.6%). There was no obvious relation between CNC word score and the PLV for each of the testing conditions. This observation was confirmed by the result of a Pearson product-moment correlation test with Bonferroni correction for multiple testing (Quiet: r=−0.18, p=0.193; +10 dB SNR: r=−0.08, p=0.353; 429+5 dB SNR: r=0.15, p=0.238).

A careful inspection of study results showed that the amount of change in CNC word scores with the presence of noise also varied among CI users (+10 dB SNR: range: -32-4.0%, mean:-15.0%, SD:-9.5%; +5 dB SNR: range:-46.0--6.0%, mean:-27.4%, SD:-10.9%). For individual participants, the amount of change could not be predicted based on their scores measured in quiet. For example, the CNC scores measured in quiet in the right car of participants A5 (A5R) and A19 (A19R) were 88% and 84%, respectively. While A19R showed a 44% decrease in CNC word score when a noise at +5 dB SNR was added, A5R only had a 6% decrease. Similarly, a CNC score of 72% measured in quiet was obtained for A3L and A15R. While A3L showed a 12% decrease in CNC word score, A15R had a 42% decrease when a noise at +5 dB SNR was added. Finally, both A7R and A17L showed a 32% decrease in CNC word scores when a noise at +5 dB SNR was added despite a 56% difference in CNC word scores measured in quiet between these two cases (scores measured in quiet: A7R: 96%, A17L: 40%). These observations were confirmed by the results of Pearson product-moment correlation tests with Bonferroni correction for multiple testing showing the nonsignificant correlation between CNC word score measured in quiet and the change in CNC word score when noise was added (+10 dB SNR: r=−0.07, p =0.365; +5 dB SNR: r=−0.18, p=0.192). Due to these variations in the amount of change in CNC word scores with the presence of noise, participants with similar scores measured in quiet could show largely different scores measured in noise and vice versa. Overall, these results suggested individual variations in susceptibility to background noise among CI users, which could not be fully captured by their scores measured in noise.

To determine whether neural synchrony in the CN was a potential contributing factor to individual variations in noise susceptibility, the relation between the PLV and the degree of noise effect on CNC word scores was evaluated, which was quantified as the amount of change in CNC word scores with the presence of noise.

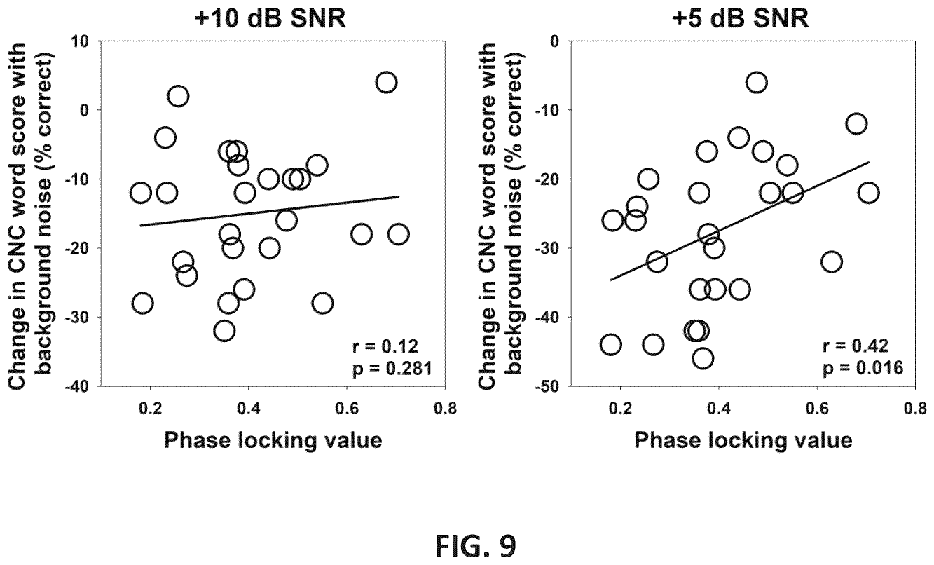

FIG. 9 shows the change in 457 CNC word scores plotted as a function of the PLV for both noise conditions. There was no obvious relation between the noise effect on CNC word score and the PLV for the +10 dB SNR noise condition, which was confirmed by the result of a Pearson product-moment correlation test (r=−0.12, p=0.281). The result of a Pearson product-moment correlation test with Bonferroni correction for multiple testing showed a moderate, negative correlation between the PLV and the degree of detrimental effect of background noise on CNC word score for the results measured at a SNR of +5 dB (r=−0.42, p=0.016), with larger PLVs associated with smaller negative effects of background noise. This correlation is statistically significant after adjusting for multiple comparisons.

### I. Computing Environment

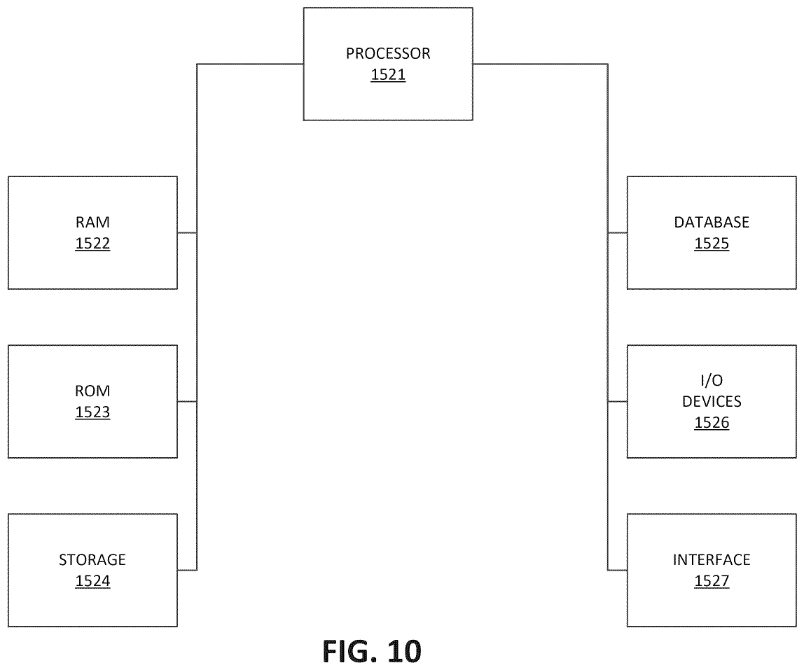

The above-described methods may be implemented on a computing system. The system has been described above as comprised of units. One skilled in the art will appreciate that this is a functional description and that the respective functions can be performed by software, hardware, or a combination of software and hardware. A unit can be software, hardware, or a combination of software and hardware. The units can comprise software for methods of removing stimulation artifacts from physiological recordings after single and multi-pulses. In one exemplary aspect, the units can comprise a computing device that comprises a processor 921 as illustrated in FIG. 10 and described below.

FIG. 10 illustrates an exemplary computer that can be used for determining a peripheral neural synchrony value based on electrically evoked compound action potential (eCAP) and correlating the peripheral neural synchrony value with temporal resolution acuity and speech perception outcomes. As used herein, “computer” may include a plurality of computers, a processor, a controller, and/or a plurality of processors and/or controllers. The computers may include one or more hardware components such as, for example, a processor 1521, a random-access memory (RAM) module 1522, a read-only memory (ROM) module 1523, a storage 1524, a database 1525, one or more input/output (I/O) devices 1526, and an interface 1527. All of the hardware components listed above may not be necessary to practice the methods described herein. Alternatively and/or additionally, the computer may include one or more software components such as, for example, a computer-readable medium including computer executable instructions for performing a method associated with the exemplary embodiments. It is contemplated that one or more of the hardware components listed above may be implemented using software. For example, storage 1524 may include a software partition associated with one or more other hardware components. It is understood that the components listed above are exemplary only and not intended to be limiting.

Processor 1521 may include one or more processors, each configured to execute instructions and process data to perform one or more functions associated with determining a peripheral neural synchrony value based on eCAPS; and correlating the peripheral neural synchrony value with temporal resolution acuity and speech perception outcomes measured in quiet and in noise in the patient. Processor 1521 may be communicatively coupled to RAM 1522, ROM 1523, storage 1524, database 1525, I/O devices 1526, and interface 1527. Processor 1521 may be configured to execute sequences of computer program instructions to perform various processes. The computer program instructions may be loaded into RAM 1522 for execution by processor 1521.

RAM 1522 and ROM 1523 may each include one or more devices for storing information associated with operation of processor 1521. For example, ROM 1523 may include a memory device configured to access and store information associated with the computer, including information for identifying, initializing, and monitoring the operation of one or more components and subsystems. RAM 1522 may include a memory device for storing data associated with one or more operations of processor 1521. For example, ROM 1523 may load instructions into RAM 1522 for execution by processor 1521.

Storage 1524 may include any type of mass storage device configured to store information that processor 1521 may need to perform processes consistent with the disclosed embodiments. For example, storage 1524 may include one or more magnetic and/or optical disk devices, such as hard drives, CD-ROMs, DVD-ROMs, or any other type of mass media device.

Database 1525 may include one or more software and/or hardware components that cooperate to store, organize, sort, filter, and/or arrange data used by the computer and/or processor 1521. For example, database 1525 may store raw data, as described herein and computer-executable instructions for determining a peripheral neural synchrony value based on eCAPS; and correlating the peripheral neural synchrony value with temporal resolution acuity and speech perception outcomes measured in quiet and in noise in the patient. It is contemplated that database 1525 may store additional and/or different information than that listed above.

I/O devices 1526 may include one or more components configured to communicate information with a user associated with computer. For example, I/O devices may include a console with an integrated keyboard and mouse to allow a user to maintain a database of data related to determining a peripheral neural synchrony value based on eCAPS; correlating the peripheral neural synchrony value with temporal resolution acuity and speech perception outcomes measured in quiet and in noise in the patient, and the like. I/O devices 1526 may also include a display including a graphical user interface (GUI) for outputting information on a monitor. I/O devices 1526 may also include peripheral devices such as, for example, a printer for printing information associated with the computer, a user-accessible disk drive (e.g., a USB port, a floppy, CD-ROM, or DVD-ROM drive, etc.) to allow a user to input data stored on a portable media device, a microphone, a speaker system, or any other suitable type of interface device.

Interface 1527 may include one or more components configured to transmit and receive data via a communication network, such as the Internet, a local area network, a workstation peer-to-peer network, a direct link network, a wireless network, or any other suitable communication platform. For example, interface 1527 may include one or more modulators, demodulators, multiplexers, demultiplexers, network communication devices, wireless devices, antennas, modems, and any other type of device configured to enable data communication via a communication network.

### II. Conclusion

While the methods and systems have been described in connection with preferred embodiments and specific examples, it is not intended that the scope be limited to the particular embodiments set forth, as the embodiments herein are intended in all respects to be illustrative rather than restrictive.

Unless otherwise expressly stated, it is in no way intended that any method set forth herein be construed as requiring that its steps be performed in a specific order. Accordingly, where a method claim does not actually recite an order to be followed by its steps or it is not otherwise specifically stated in the claims or descriptions that the steps are to be limited to a specific order, it is no way intended that an order be inferred, in any respect. This holds for any possible non-express basis for interpretation, including: matters of logic with respect to arrangement of steps or operational flow; plain meaning derived from grammatical organization or punctuation; the number or type of embodiments described in the specification.

Throughout this application, various publications may be referenced. The disclosures of these publications in their entireties are hereby incorporated by reference into this application in order to more fully describe the state of the art to which the methods and systems pertain.

The publications incorporated by reference include, but are not limited to, the following:
A. He, S., Teagle, H. F., & Buchman, C. A. (2017). The electrically evoked compound action potential: from laboratory to clinic. Frontiers in neuroscience, 11, 339.
B. He S, Skidmore J, Bruce I C, Oleson J J, Yuan Y. Peripheral neural synchrony in post-lingually deafened adult cochlear implant users. medRxiv [Preprint]. 2024 Feb. 16:2023.07.07.23292369. doi: 10.1101/2023.07.07.23292369. Update in: Ear Hear. 2024 September-October 1;45 (5): 1125-1137. doi: 10.1097/AUD.0000000000001502. PMID: 37461681; PMCID: PMC10350140.

It will be apparent to those skilled in the art that various modifications and variations can be made without departing from the scope or spirit. Other embodiments will be apparent to those skilled in the art from consideration of the specification and practice disclosed herein. It is intended that the specification and examples be considered as exemplary only, with a true scope and spirit being indicated by the following claims.

What is claimed is:

1. A method of determining a peripheral neural synchrony value based on eCAPS and correlating the peripheral neural synchrony value with temporal resolution acuity and speech perception outcomes comprising:
(a) providing a stimulus at a first of electrode of a cochlear implant (CI) of a patient, wherein the stimulus is set at or below a comfort level of the patient;
(b) measuring eCAP at a second electrode of the CI of the patient, said eCAP based on a neural response to the stimulus;
(c) repeating steps (a) and (b) sequentially a plurality of times for a plurality of electrodes of the CI to obtain a plurality of eCAP measurements;
(d) evaluating a phase coherence among the plurality of eCAP measurements to determine a peripheral neural synchrony value; and
(e) correlating the peripheral neural synchrony value with temporal resolution acuity and speech perception outcomes measured in quiet and in noise in the patient.

2. The method of claim 1, wherein the neural response to the stimulus is obtained by sending a user-defined stimuli through the first electrode of the cochlear implant to stimulate surrounding neurons and recording an electrical response of the surrounding electrons using the second electrode of the cochlear implant.

3. The method of claim 1, wherein the peripheral neural synchrony value is quantified using a phase locking value (PLV) index, which is a measure of the phase coherence among the plurality of eCAP measurements.

4. The method of claim 3, wherein larger PLVs indicate better or stronger neural synchrony in a cochlear nerve (CN) of the patient, wherein the PLV is a length of a vector formed by averaging complex phase angles of the plurality of eCAP measurements at individual frequencies obtained via time-frequency decomposition.

5. The method of claim 4, wherein the PLV is calculated at a specific frequency and time window (i.e., frame) as:
$$PLV ( f , t ) = \frac{1}{400} {\sum }_{k = 1}^{400} \vert \frac{{F}_{k} ( f , t )}{\vert {F}_{k} ( f , t ) \vert} \vert$$
where Fk(f,t) is a spectral estimate (i.e., a complex number representing an amplitude and a phase of a sinusoid obtained from a short-time Fourier transform) of eCAP measurement k at frequency f for time window t.

6. The method of claim 1, wherein speech perception outcomes are evaluated separately for each ear of the patient having a cochlear implant.

7. The method of claim 6, wherein the speech perception outcomes are evaluated using Consonant-Nucleus-Consonant (CNC) word lists presented in quiet and in one or more noise conditions.

8. A system for determining a peripheral neural synchrony value based on eCAPS and correlating the peripheral neural synchrony value with temporal resolution acuity and speech perception outcomes comprising:
a memory; and
a processor in communication with the memory, wherein the processor executes computer-executable instructions stored in the memory, said instructions causing the processor to:
(a) provide a stimulus at a first of electrode of a cochlear implant (CI) of a patient, wherein the stimulus is set at or below a comfort level of the patient;
(b) measure eCAP at a second electrode of the CI of the patient, said eCAP based on a neural response to the stimulus;
(c) repeat steps (a) and (b) sequentially a plurality of times for a plurality of electrodes of the CI to obtain a plurality of eCAP measurements;
(d) evaluate a phase coherence among the plurality of eCAP measurements to determine a peripheral neural synchrony value; and
(e) correlate the peripheral neural synchrony value with temporal resolution acuity and speech perception outcomes measured in quiet and in noise in the patient.

9. The system of claim 8, wherein the neural response to the stimulus is obtained by sending a user-defined stimuli through the first electrode of the cochlear implant to stimulate surrounding neurons and recording an electrical response of the surrounding electrons using the second electrode of the cochlear implant.

10. The system of claim 8, wherein the peripheral neural synchrony value is quantified using a phase locking value (PLV) index, which is a measure of the phase coherence among the plurality of eCAP measurements, wherein larger PLVs indicate better or stronger neural synchrony in a cochlear nerve (CN) of the patient, wherein the PLV is a length of a vector formed by averaging complex phase angles of the plurality of eCAP measurements at individual frequencies obtained via time-frequency decomposition.

11. The system of claim 10, wherein the PLV is calculated at a specific frequency and time window (i.e., frame) as:
$$PLV ( f , t ) = \frac{1}{400} {\sum }_{k = 1}^{400} \vert \frac{{F}_{k} ( f , t )}{\vert {F}_{k} ( f , t ) \vert} \vert$$
where Fk(f,t) is a spectral estimate (i.e., a complex number representing an amplitude and a phase of a sinusoid obtained from a short-time Fourier transform) of eCAP measurement k at frequency f for time window t.

12. The system of claim 8, wherein speech perception outcomes are evaluated separately for each ear of the patient having a cochlear implant.

13. The system of claim 12, wherein the speech perception outcomes are evaluated using Consonant-Nucleus-Consonant (CNC) word lists presented in quiet and in one or more noise conditions.

14. A computer-program product comprising computer-executable instructions stored on a non-transitory medium, said computer-executable instructions for performing a determining a peripheral neural synchrony value based on eCAPS and correlating the peripheral neural synchrony value with temporal resolution acuity and speech perception outcomes, said method comprising:
(a) providing a stimulus at a first of electrode of a cochlear implant (CI) of a patient, wherein the stimulus is set at or below a comfort level of the patient;
(b) measuring eCAP at a second electrode of the CI of the patient, said eCAP based on a neural response to the stimulus;
(c) repeating steps (a) and (b) sequentially a plurality of times for a plurality of electrodes of the CI to obtain a plurality of eCAP measurements;
(d) evaluating a phase coherence among the plurality of eCAP measurements to determine a peripheral neural synchrony value; and
(e) correlating the peripheral neural synchrony value with temporal resolution acuity and speech perception outcomes measured in quiet and in noise in the patient.

15. The computer-program product of claim 14, wherein the neural response to the stimulus is obtained by sending a user-defined stimuli through the first electrode of the cochlear implant to stimulate surrounding neurons and recording an electrical response of the surrounding electrons using the second electrode of the cochlear implant.

16. The computer-program product of claim 14, wherein the peripheral neural synchrony value is quantified using a phase locking value (PLV) index, which is a measure of the phase coherence among the plurality of eCAP measurements.

17. The computer-program product of claim 16, wherein larger PLVs indicate better or stronger neural synchrony in a cochlear nerve (CN) of the patient, wherein the PLV is a length of a vector formed by averaging complex phase angles of the plurality of eCAP measurements at individual frequencies obtained via time-frequency decomposition.

18. The computer-program product of claim 17, wherein the PLV is calculated at a specific frequency and time window (i.e., frame) as:
$$PLV ( f , t ) = \frac{1}{400} {\sum }_{k = 1}^{400} \vert \frac{{F}_{k} ( f , t )}{\vert {F}_{k} ( f , t ) \vert} \vert$$
where Fk(f,t) is a spectral estimate (i.e., a complex number representing an amplitude and a phase of a sinusoid obtained from a short-time Fourier transform) of eCAP measurement k at frequency f for time window t.

19. The computer-program product of claim 14, wherein speech perception outcomes are evaluated separately for each ear of the patient having a cochlear implant.

20. The computer-program product of claim 19, wherein the speech perception outcomes are evaluated using Consonant-Nucleus-Consonant (CNC) word lists presented in quiet and in one or more noise conditions.
